(19) United States
(12) Patent Application Publication
(10) Pub. No.: US 2010/0145306 A1
(43) Pub. Date: Jun. 10, 2010

(54) Various Catheter Devices for Myocardial Injections or Other Uses

(75) Inventors: Timothy J. Mickley, Corcoran, MN (US); Grace Kim, Minneapolis, MN (US); Samuel J. Epstein, Marlborough, MA (US); Mark Griffin, Louisville, KY (US); John Edrington, Jefferson, IN (US); Greg Furnish, Louisville, KY (US)

Correspondence Address: VIDAS, ARRETT & STEINKRAUS, P.A., SUITE 400, 6640 SHADY OAK ROAD, EDEN PRAIRIE, MN, 55344, US

(73) Assignee: Boston Scientific Scimed, Inc., Maple Grove, MN (US)

(21) Appl. No.: 12/509,105

(22) Filed: Jul. 24, 2009

Related U.S. Application Data
(60) Provisional application No. 61/083,314, filed on Jul. 24, 2008.

(51) Int. Cl. A61M 25/06; A61M 25/00

(52) U.S. Cl. 604/508; 604/523; 604/529

(57) ABSTRACT

A catheter device comprising a proboscis shaft and a proboscis disposed within the proboscis shaft. In certain embodiments, the catheter device further comprises an elongate tubular member, wherein the proboscis shaft is disposed within the elongate tubular member. In certain embodiments, the catheter device comprises a tissue surface engagement structure, which has a first configuration and a second configuration. In the second configuration, the tissue surface engagement structure presents a larger transverse profile in comparison to the first configuration. The tissue surface engagement structure may have any of various designs, including an expandable assembly and a hinged assembly. In certain embodiments, a deformable cushion is positioned at the distal end of the proboscis shaft. The deformable cushion comprises a pocket that is filled with a reshapeable material. In certain embodiments, the proboscis shaft comprises a longitudinally compressible portion.

### TECHNICAL FIELD

The present invention relates to medical devices, more particularly, to catheter devices.

### BACKGROUND

Catheters are used in a wide variety of minimally-invasive or percutaneous medical procedure. One type of catheter is an intravascular, which enables a physician to remotely perform a medical procedure by inserting the catheter into the vascular system of the patient at an easily accessible location and navigating the tip of the catheter to the target site. Using catheter-guided methods, many internal sites may be remotely accessed through the patient's vascular system or other body lumen structure.

In some applications, a needle may be connected to a catheter assembly to deliver a therapeutic agent into remote sites within a patient's body. For example, in a percutaneous myocardial revascularization procedure, the inside surface of the heart is accessed by an intravascular catheter via a retrograde route though the arterial system. A needle is advanced through the catheter, and the heart muscle is then injected with therapeutic agents, such as stem cells or drugs, to promote new blood vessel formation in the heart muscle.

However, the use of an injection catheter can cause injury to the myocardium, which in the most serious cases, results in myocaridial wall perforation. One the of the possible causes of injury is the distal tip of the delivery catheter. Therefore, it is desirable to provide a catheter device that can deliver therapeutic or diagnostic agents to the myocardium while reducing the risk of traumatic injury.

### SUMMARY

In one aspect, the present invention provides a catheter device comprising: (a) a proboscis shaft having a lumen and an exit opening at the distal end of the proboscis shaft: (b) a tissue surface engagement structure positioned at the distal end of the proboscis shaft, wherein the tissue surface engagement structure has a first configuration and a second configuration, and wherein the transverse profile of the tissue surface engagement structure is larger in the second configuration than in the first configuration; and (c) a proboscis disposed within the lumen of the proboscis shaft.

In another aspect, the present invention provides a catheter device comprising: (a) a proboscis shaft having a lumen and an exit opening at the distal end of the proboscis shaft: (b) a deformable cushion positioned at the distal end of the proboscis shaft, wherein the cushion has a pocket filled with a reshapeable material, and wherein the cushion has a contact surface for engaging a target site; and (c) a proboscis disposed within the lumen of the proboscis shaft.

In another aspect, the present invention provides a catheter device comprising: (a) a proboscis shaft having a longitudinally compressible portion, a lumen, and an exit opening at the distal end of the proboscis shaft; and (b) a proboscis disposed within the lumen of the proboscis shaft.

In other aspects, the present invention provides methods for delivering a therapeutic or diagnostic agent into myocardium by using catheter devices of the present invention.

### BRIEF DESCRIPTION OF THE DRAWINGS

FIGS. 1A and 1B show side views in partial cross-section and FIG. 1C shows a perspective view of the distal portion of a catheter device according to an embodiment of the present invention.

FIGS. 2A and 2B show side views in partial cross-section and FIG. 2C shows a perspective view of the distal portion of a catheter device according to another embodiment. FIGS. 2D and 2E show distal end views of the proboscis shaft of the catheter device.

FIG. 3A shows a perspective view of the distal portion of a catheter device according to yet another embodiment. FIGS. 3B and 3C show distal end views of the proboscis shaft of the catheter device.

FIGS. 4A and 4B show side views in partial cross-section of the distal portion of a catheter device according to yet another embodiment.

FIGS. 5A and 5B show side views of the distal portion of a catheter device according to yet another embodiment.

FIGS. 6A and 6B show side views of the distal portion of a catheter device according to yet another embodiment.

FIGS. 7A and 7B show side views in partial cross-section of the distal portion of a catheter device according to yet another embodiment.

FIGS. 8A and 8B show side views of the distal portion of a catheter device according to yet another embodiment.

FIGS. 9A and 9B show side views of the distal portion of a catheter device according to yet another embodiment.

FIGS. 10A and 10B show side views of the distal portion of a catheter device according to yet another embodiment.

### DETAILED DESCRIPTION

A catheter device of the present comprises a proboscis shaft and a proboscis disposed within the proboscis shaft. As used herein, the term “proboscis” refers to an elongate structure that contacts or penetrates into tissue to provide and/or deliver a diagnostic or therapeutic intervention. Examples of proboscises include injection needles; injection catheters; electrodes; sensors; probes including those used for applying RF or microwave therapy, cryotherapy, or ultrasound; or optical fibers (e.g., for use in sensing, imaging, phototherapy, or laser ablation therapy, such as in transmyocarial revascularization). Depending upon the particular application, the proboscis may have any of various configurations or characteristics; for example, the proboscis may be curved or straight, hollow or solid, sharp or blunt.

The proboscis shaft is a tubular structure having a lumen for containing a proboscis. At its distal end, the proboscis shaft also has an exit opening to allow the proboscis to exit from the proboscis shaft. The proboscis is disposed within the lumen of the proboscis shaft may be telescopically slidable in relation to the proboscis shaft. As such, the proboscis may be retracted within the proboscis shaft and then advanced so that the distal end of the proboscis exits from the exit opening of the proboscis shaft. As used herein, the terms “advanced” and “retracted,” when referring to the proboscis and the proboscis shaft, are intended to refer to relative motion between the two elements such that the proboscis moves distally in relation to the proboscis shaft (the proboscis is advanced) or the proboscis moves proximally in relation to the proboscis shaft (the proboscis is retracted). As such, advancing the proboscis may be carried out by moving the proboscis distally or by moving the proboscis shaft proximally. Likewise, retracting the proboscis may be carried out by moving the proboscis proximally or by moving the proboscis shaft distally. The proboscis shaft functions to guide and/or deliver the proboscis to the target site, and as such, the proboscis shaft may be part of a delivery catheter.

In some embodiments, the catheter device may further comprise an elongate tubular member having a lumen for containing the proboscis shaft. At its distal end, the elongate tubular member has an exit opening to allow the proboscis shaft to exit from the elongate tubular member. The proboscis shaft is disposed within the lumen of the elongate tubular member and may be telescopically slidable in relation to the elongate tubular member. As such, the proboscis shaft may be retracted within the elongate tubular member and advanced so that the distal end of the proboscis shaft exits from the exit opening of the elongate tubular member. As used herein, the terms “advanced” and “retracted”, when referring to the proboscis shaft and the elongate tubular member, are intended to refer to relative motion between the two elements such that the proboscis shaft moves distally in relation to the elongate tubular member (the proboscis shaft is advanced) or the proboscis shaft moves proximally in relation to the elongate tubular member (the proboscis shaft is retracted). As such, advancing the proboscis shaft may be carried out by moving the proboscis shaft distally or by moving the elongate tubular member proximally. Likewise, retracting the proboscis shaft may be carried out by moving the proboscis shaft proximally or by moving the elongate tubular member distally. The elongate tubular member functions to guide and/or deliver the proboscis and the proboscis shaft to the target site, and as such, the elongate tubular member may be part of a delivery catheter.

In one aspect, a catheter device of the present invention further comprises a tissue surface engagement structure positioned at the distal end of the proboscis shaft. The proboscis shaft and the tissue surface engagement structure may form a single unitary structure or the two components may be separate units that are couples together. The tissue surface engagement structure is designed to allow the proboscis shaft to engage the surface of body tissue in such a way as to reduce the risk of injury to the tissue.

The tissue surface engagement structure has a first configuration and a second configuration, and is changeable between the two configurations. When actuated, the tissue surface engagement structure switches from the first configuration to the second configuration, and in some cases, may be reverted back to the first configuration. In the second configuration, the tissue surface engagement structure presents a larger transverse profile in comparison to the first configuration. As used herein, “transverse profile” refers to a two-dimensional representation of the tissue surface engagement structure when viewed from a point distally along the central longitudinal axis of the proboscis shaft (i.e., and image of the tissue surface engagement structure as projected onto a plane that is transverse to the central longitudinal axis). Where there are void spaces enclosed within the peripheral outline of the two-dimensional representation (e.g., the outline of a wire loop), the transverse profile includes all the area enclosed by the peripheral outline of the two-dimensional representation. In this way, in the second configuration, the tissue surface engagement structure provides a larger surface area for the proboscis shaft to engage the surface of the tissue, thereby reducing the risk of injury.

In certain embodiments, the size of the transverse profile of the tissue surface engagement structure in the second configuration is at least 1.5 times the size of the transverse profile in the first configuration. In some cases, the size of the transverse profile of the tissue surface engagement structure in the second configuration is 1.5 to 10 times the size; and in some cases, 1.5 to 5 times the size; and in some cases, 1.5 to 3 times the size of the transverse profile in the first configuration. Other rangers are also possible, depending upon the particular application. The amount of increase in the transverse profile will depend upon various factors, including the size, shape, and dimensions of the catheter device; the materials used to make the catheter device; how the catheter device operates; the type of procedure being performed; and the type of tissue being engaged. For example, a catheter device for use on softer, more fragile body tissue may need a larger increase in the transverse profile than a catheter device for use on more durable body tissue.

Actuation of the tissue surface engagement structure may be controlled using any of various mechanisms, including mechanical (e.g., using levers, wires, strings, pulleys, plungers, etc.), electrical, electro-mechanical, chemical pneumatic, or hydraulic mechanisms. In some cases, the tissue surface engagement structure is self-actuated, which can be provided by designing the tissue surface engagement structure to be biased towards the first configuration or the second configuration. For example, the tissue surface engagement structure can be designed with a bias towards one configuration by using shape memory material such as nitinol, stainless steel, other super-elastic metal alloys, or polymeric materials. In some cases, the tissue surface engagement structure can be actuated by pressing it against the target tissue surface.

The tissue surface engagement structure may have any of various designs, with first and second configurations, that are suitable for performing the function of engaging a tissue surface. The particular design of the tissue surface engagement structure will depend on various factors, such as the size, shape, and dimensions of the catheter device; the materials used to make the catheter device; how the catheter device operates; the type of procedure being performed; and the type of tissue being engaged. For example, the tissue surface engagement structure may be an expandable assembly or a hinged assembly.

In certain embodiments, the tissue surface engagement structure is an expandable assembly. In the first configuration, the expandable assembly is in a collapsed configuration. In the second configuration, the expandable assembly is in an expanded configuration. The expansion occurs at least partially in a radial direction relative to the central longitudinal axis of the proboscis shaft. In the expanded configuration, the expandable assembly presents a larger transverse profile. As explained above, various amounts of increase in the transverse profile are possible.

The expandable assembly may be a single unitary structure or it may comprise one or more subunits that engage the tissue surface. The expandable assembly may have any of various possible designs and may be made from any of various types of materials. For example, the expandable assembly may be a wire basket, a wire mesh, a balloon, a canopy, or an umbrella; or it may comprise one or more loops, petals, tabs, strips, or sleeves. As described above, the expandable assembly can be actuated in various ways, including self-actuation (i.e., the expandable assembly is self-expandable or self-folding).

In embodiments where the catheter device further comprises an elongate tubular member, with the proboscis shaft disposed within the elongate tubular member, the expandable assembly may be in a collapsed configuration when the proboscis shaft is retracted within the elongate tubular member. When the proboscis shaft is advanced out of the elongate tubular member, the expandable assembly is expanded to its expanded configuration.

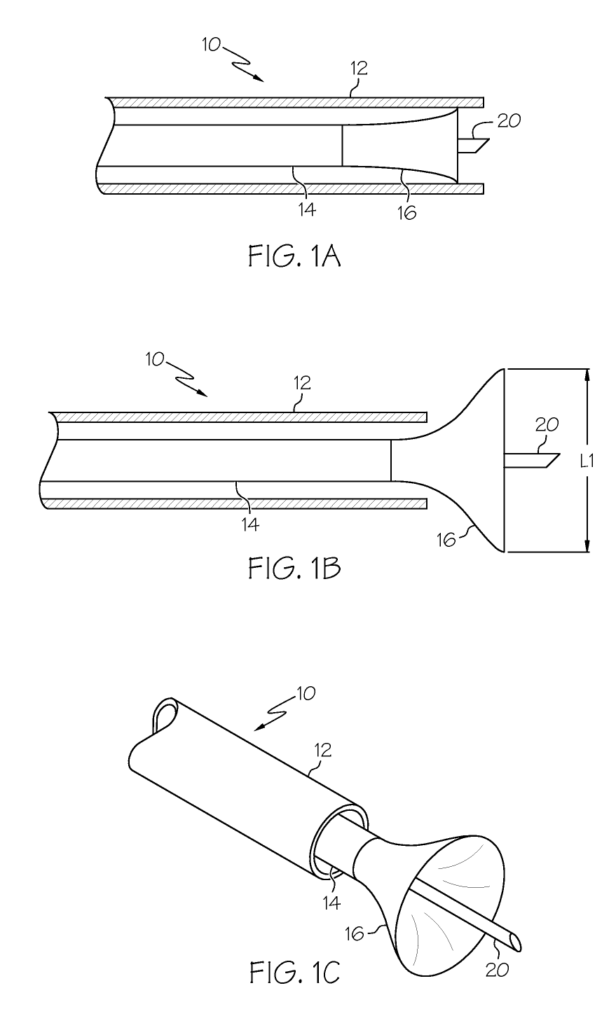

The following non-limiting examples further illustrate various embodiments of the present invention. Referring to FIGS. 1A-1C, a catheter 10 comprises an elongate tubular member 12, a proboscis shaft 14, a proboscis in the form of an injection needle 20, and self-expandable canopy 16 made of a shape memory or resilient material. Referring to FIG. 1A, when proboscis shaft 14 is retracted, canopy 16 is maintained in a collapsed configuration within elongate tubular member 12. Referring to FIG. 1B, when proboscis shaft 14 is advanced, resilient bias causes canopy 16 to an expanded configuration. The diameter (L1) of canopy 16 in its expanded configuration may be in the range of 0.040 inches to 0.090 inches, but other diameters are also possible, depending upon the particular application. Canopy 16 may also be further expanded by compressing it against the tissue surface.

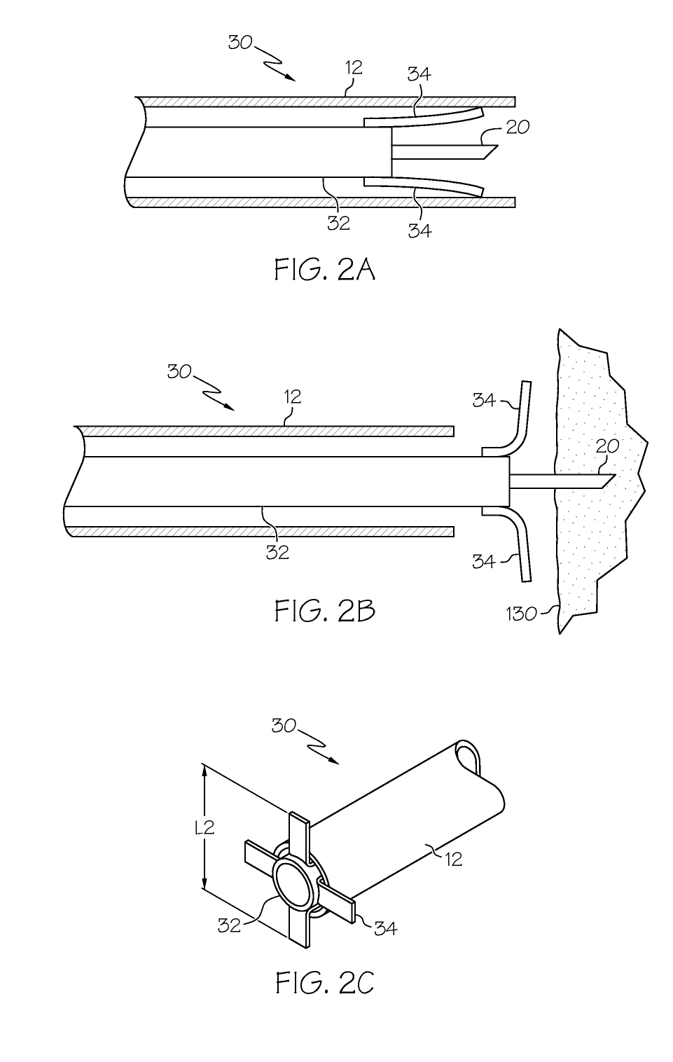

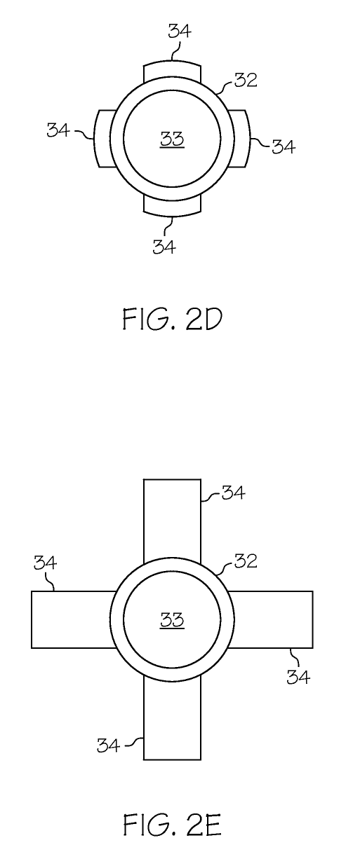

In another embodiment, referring to FIGS. 2A-2E, a catheter device 30 comprises elongate tubular member 12, a proboscis shaft 32, injection needle 20, and a self-expandable petal tab structure having four petal tabs 34. Referring to FIG. 2A (shown without the laterally positioned petal tabs), when proboscis shaft 32 is retracted, petal tabs 34 are unbent such that the petal tab structure is maintained in a collapsed configuration. Referring to FIG. 2B (shown without the laterally positioned petal tabs), when proboscis shaft 32 is advanced, petal tab structure self-expands to an expanded configuration by petal tabs 34 bending outward. Here, petal tabs 34 form an angle that is substantially or nearly orthogonal to the central longitudinal axis of proboscis shaft 32. The end-to-end span (L2) of the petal tab structure may be in the range of 0.040 inches to 0.090 inches, but others ranges are also possible, depending upon the application.

FIG. 2B also shows the operation of catheter device 30 when used for myocardial injections. The distal end of the catheter device 30 is positioned in an internal chamber of the heart and is guided to approach the myocardial wall 130. The petal structure is expanded and made to engage the myocardial wall 130. Injection needle 20 is made to penetrate myocardial wall 130, which may, in some cases, be performed by advancing injection needle 20. Penetration of myocardial wall 130 by injection needle 20 may occur before, after, or simultaneous with the petal tab structure engaging the myocardial wall 130. A therapeutic or diagnostic agent is then delivered to the myocardium through injection needle 20.

FIGS. 2D and 2E show distal views of proboscis shaft 32 (with its lumen 33) and demonstrate the transverse profiles of the petal tab structure. FIG. 2D shows the petal tab structure in a collapsed configuration, and FIG. 2E shows petal tab structure in the expanded configuration. This demonstrates that the transverse profile of the petal tab structure in the expanded configuration is larger than its transverse profile in the collapsed configuration.

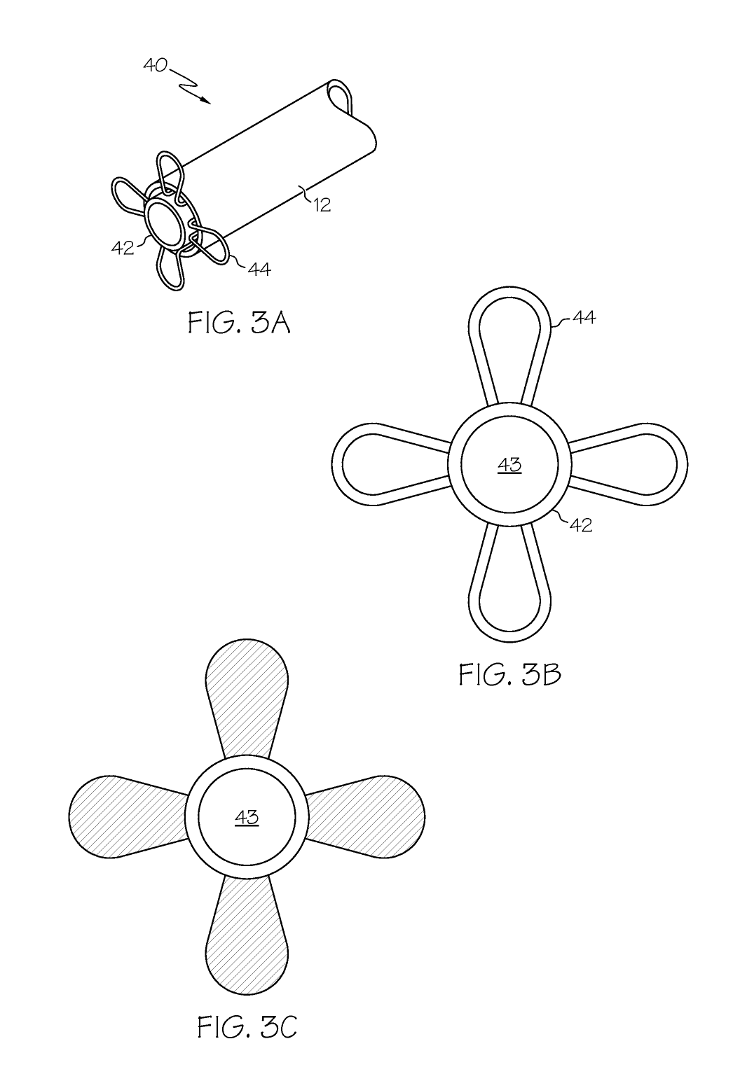

In yet another embodiment, referring to FIG. 3A, a catheter device 40 comprises elongate tubular member 12, a proboscis shaft 42, an injection needle (not shown), and a self-expandable petal loop structure having four petal loops 44. Similar to catheter device 30 shown in FIGS. 2A-2E, when proboscis shaft 42 is retracted, petal loops 44 are unbent such that the petal loop structure is maintained in a collapsed configuration within elongate tubular member 12. When proboscis shaft 42 is advanced, the petal loop structure self-expands to an expanded configuration by petal loops 44 bending outward. FIG. 3B shows the distal end view of proboscis shaft 42 (with its lumen 43), demonstrating the transverse profile of the petal loop structure in its expanded configuration. As shown in FIG. 3C, the transverse profile of the petal loops includes all the area (in cross-hatch) enclosed within the peripheral outline of the petal loops.

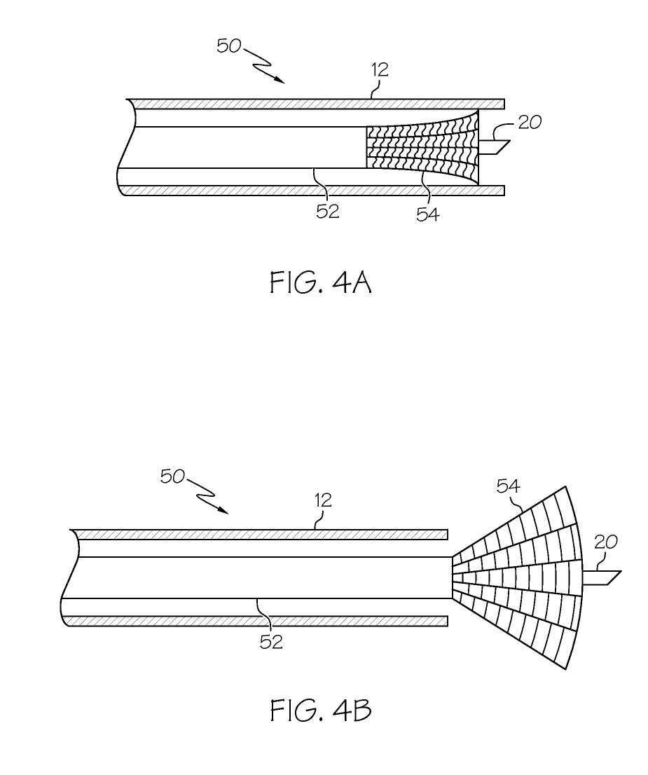

In yet another embodiment, referring to FIGS. 4A and 4B, a catheter device 50 comprises elongate tubular member 12, a proboscis shaft 52, injection needle 20, and self-expandable wire mesh structure 54. Referring to FIG. 4A, when proboscis shaft 52 is retracted, mesh structure 54 is maintained in a collapsed configuration within elongate tubular member 12. Referring to FIG. 4B, when proboscis shaft 52 is advanced, wire mesh structure 54 self-expands to an expanded configuration.

In certain embodiments, the tissue surface engagement structure is a hinged assembly comprising one or more hinged members that are hingedly joined to the proboscis shaft at one or more hinge portions. In its first configuration, the hinged assembly is in a closed configuration. In its second configuration, the hinged assembly is in an open configuration. In the open configuration, the hinged assembly presents a larger transverse profile. As explained above, various amounts of increase in the transverse profile are possible.

The hinged assembly alternates between the closed and open configurations by pivoting of the hinged members at their respective hinge portions. A hinged potion may comprise any of various types of hinges known in the art, including those using pins, leaves, springs, pivots, etc. In some cases, a hinge portion may simply be a flexible point or segment on the proboscis shaft where a hinged member joins the proboscis shaft.

In embodiments where the catheter device further comprises an elongate tubular member, with the proboscis shaft disposed within the elongate tubular member, the hinged assembly may be maintained in a closed configuration when the proboscis shaft is retracted within the elongate tubular member. When the proboscis shaft is advanced out of the elongate tubular member, the hinged assembly changes to an open configuration.

As described above, the hinged assembly can be actuated in various ways, including self-actuation (i.e. the hinged assembly is self-opening or self-closing). For example, the hinge portion may comprise a spring that biases the hinged assembly towards an open configuration. The hinged assembly may also be actuated by compressing the hinged members against the tissue surface, causing the hinged members to pivot at their respective hinge portions.

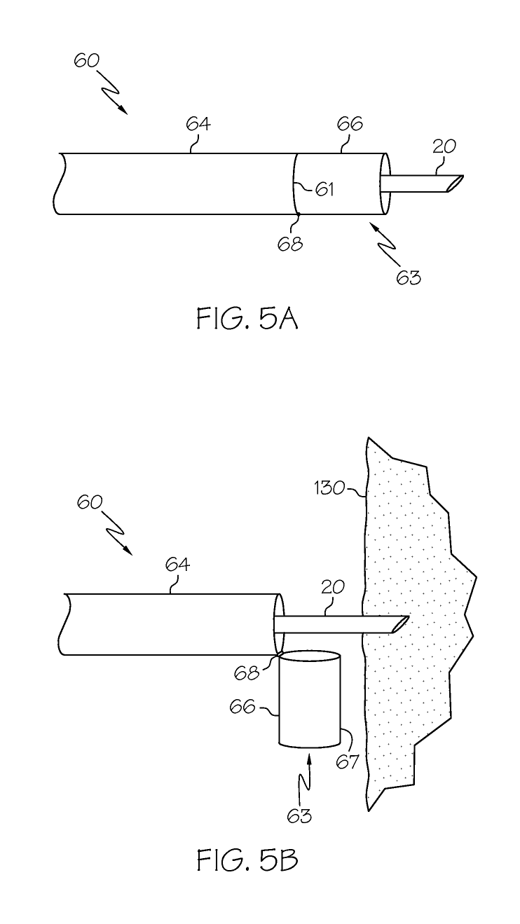

The following non-limiting examples further illustrate various embodiments of the present. Referring to FIGS. 5A and 5B, a catheter device 60 comprising a proboscis shaft 64, an injection needle 20, and a hinged assembly 63 comprising a single hinged member 66. In this embodiment, hinged member 66 is created by partially transecting the distal end of proboscis shaft 64 at seam 61. A small portion of proboscis shaft 64 is left to serve as a flexible hinge 68, which allows hinged member 66 to pivot in relation to proboscis shaft 64.

FIG. 5A shows hinged assembly 63 in a closed configuration with hinged member 66 in axial alignment with proboscis shaft 64. FIG. 5B shows hinged assembly 63 in an open configuration with hinged member 66 rotated approximately 90° so that hinged assembly 63 presents a larger transverse profile. In this embodiment, hinged assembly 63 is actuated from the closed configuration to the open configuration by compressing hinged member 66 against the target tissue surface, causing surface, causing hinged member 66 to pivot at flexible hinge 68.

FIG. 5B also shows the operation of catheter device 60 when used for myocardial injections. The distal end of catheter device 60 is positioned in an internal chamber of the heart and is guided to approach the myocardial wall 130. Hinged assembly 63 is opened and a contact surface 67 on hinged member 66 is made to engage the myocardial wall 130. Injection needle 20 is made to penetrate myocardial wall 130, which may, in some cases, be performed by advancing injection needle 20. Penetration of myocardial wall 130 by injection needle 20 may occur before, after, or simultaneous with hinged member 66 engaging the myocardial wall 130. A therapeutic or diagnostic agent is then delivered to the myocardium through injection needle 20.

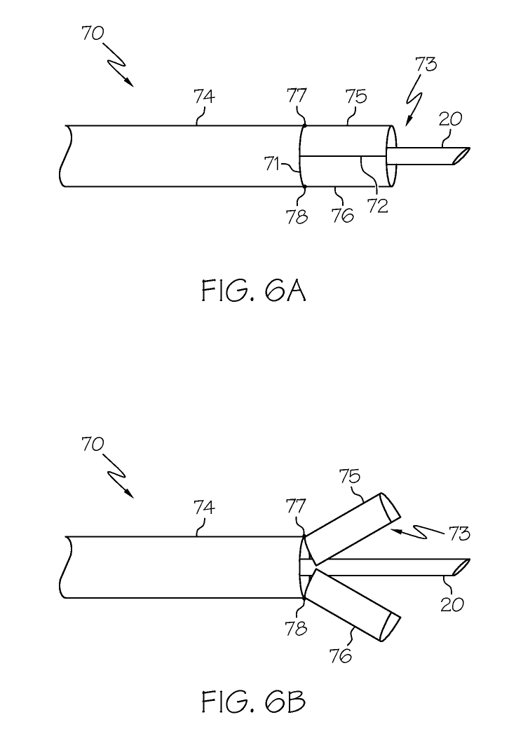

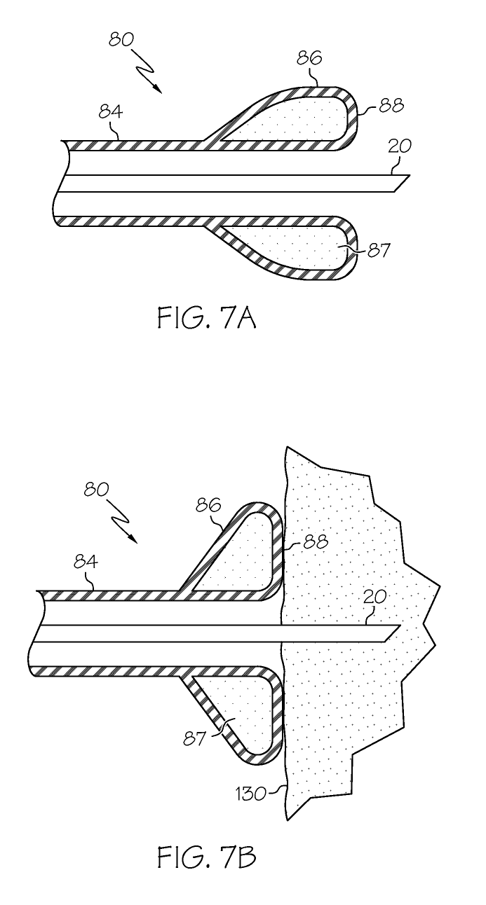

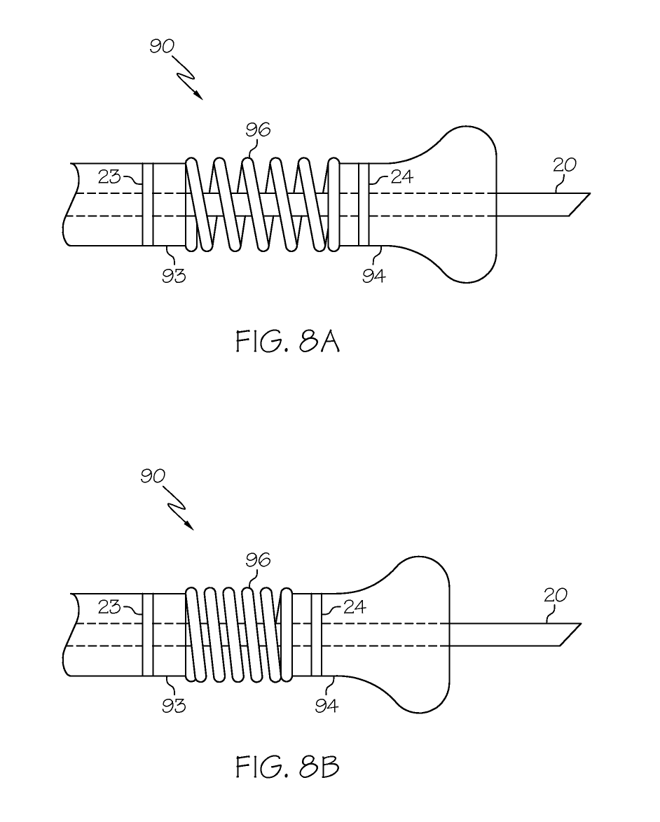

Referring to FIGS. 6A and 6B, a catheter device 70 comprises a proboscis shaft 74, an injection needle 20, and a hinged assembly 73 comprising two hinged members 75 and 76. In this embodiment, hinged members 75 and 76 are created by splitting proboscis shaft 74 at seam 72 and making partial transverse cuts at seam 71. Small portions of proboscis shaft 74 are left to serve as flexible hinges 77 and 78, which allow hinged members 75 and 76 to pivot in relations to proboscis shaft 74.

FIG. 6A shows hinged assembly 73 in a closed configuration with hinged members 75 and 76 in axial alignment with proboscis shaft 74. FIG. 6B shows hinged assembly 73 in an open configuration with hinged members 75 and 76 rotated approximately 45° so that hinged assembly 73 presents a larger transverse profile. In this embodiment, hinged assembly 73 is actuated from the closed configuration to the open configuration by compressing hinged members 75 and 76 against the target tissue surface, causing hinged members 75 and 76 to pivot at flexible hinges 77 and 78.

In another aspect of the present invention, a catheter device further comprises a deformable cushion that is positioned at the distal end of the proboscis shaft. The proboscis shaft and the cushion may be a single unitary structure or the two components may be separate units that are coupled together. The cushion includes a passageway through the proboscis travels. The passageway may be any passage by which the proboscis travels through the cushion, such as a channel, a tunnel, or simply an opening in the cushion (e.g., a central hole in a doughnut-shaped cushion).

The cushion is designed to be deformable in response to compressive force which may be applied through the proboscis shaft or by the tissue surface. The term “deformable,” as used herein when referring to a cushion, is intended to mean that the cushion can be deformed under compressive forces encountered by the cushion during a myocardial injection procedure. Information about these forces, such as quantity and direction, are known or are readily available to one of ordinary skill in the art. In some cases, the cushion will substantially return to its original shape and dimensions when the compressive force is released. This feature allow the cushion to be retracted back into a delivery catheter.

The deformable cushion comprises a pocket that is filled with a reshapeable material. As used herein, “reshapeable material” refers to materials that readily change shape when acted upon by forces that are encountered during a myocardial injection procedure. Such materials include fluids, liquids, gases, gels, or foams. For example, the pocket may be filled with saline, silicone or a polyurethane foam. The pocket may be in the form of a bladder, balloon, sac, or other type of enclosure. The cushion may have any suitable shape or form, such as collar, cylinder, washer, ring, doughnut hub, sphere, etc.

The cushion has a contact surface which engages the target tissue. The contact surface may be on any aspect of the cushion, including the sides, edges, or distal face of the cushion. If the cushion does not have defined faces (such as in a sphere), the contact surface is that portion of the surface of the cushion that engages the target tissue. When the cushion deforms under compressive forces, the area of the contact surface by which the cushion engages the tissue increases, thereby reducing the contact pressure and the attendant risk of tissue injury. Various characteristics of the cushion, such as its shape, dimensions, or material composition may be adjusted to provide the desired increase in contact surface area under the compressive forces. In some cases, the contact surface of the cushion (in its undeformed state) has an area of at least 0.9 mm².

In some cases, the reshapeable material in the pocket is sufficiently viscous that the cushion deforms under a steady compressive pressure, but does not substantially deform under transient, impulse pressures. For example, the impulse pressures may be produced by the contractile force of a beating heart. This feature may be useful where the operator (e.g., a physician) relies on tactile sensation to assess wall contact. For example, because the cushion does not absorb the impulse pressures created by the beating of the heart, these forces are transmitted to the operator and signals contact with the myocardial wall.

Referring to FIGS. 7A and 7B, a catheter device 80 comprises a proboscis shaft 84, an injection needle 20, and a cushion 86 positioned at the distal end of the proboscis shaft 84, wherein cushion 86 has a pocket 87 filled with a reshapeable material. On its distal face, cushion 86 has a contact surface 88 for engaging the target tissue. FIG. 7A shows cushion 86 in an undeformed condition, and FIG. 7B shows cushion 86 in a deformed condition.

FIG. 7B also shows the operation of catheter device 80 when used for myocardial injections. The distal end of catheter device 80 is positioned in an internal chamber of the heart and is guided to approach the myocardial wall 130. Contact surface 88 is pressed against the myocardial wall 130, causing cushion 86 to deform, thereby increasing the area of contact surface 88. In certain embodiments, the area of contact surface 88 when cushion 86 is deformed under compressive forces encountered during myocardial injection procedures is at least 1.5 times the area of contact surface 88 when cushion 86 is undeformed; and in some cases, from 1.5 to 10 times the area; and in some cases, from 1.5 to 3 times the area.

Injection needle 20 is made to penetrate the myocardial wall 130. Penetration of the myocardial wall 130 by injection needle 20 may occur before, after, or simultaneous with cushion 86 engaging the myocardial wall 130. For example, injection needle 20 may be fully retracted inside proboscis shaft 84, and then exposed when cushion 86 becomes compressed. A therapeutic or diagnostic agent is then delivered to the myocardium through injection needle 20.

In another aspect of the present invention, the proboscis shaft on a catheter device comprises a longitudinally compressible portion. When the proboscis shaft is compressed against a tissue surface, the compressible portion absorbs the compressive force to reduce the amount of force applied against the tissue surface.

In response to compressive forces (such as those experienced during myocardial injection procedures), the compressible portion is designed to undergo compression in the longitudinally direction in relation to the proboscis shaft. As used herein, “longitudinally compressible” means compressibility at least in a direction parallel to the central longitudinal axis of the proboscis shaft. As such, longitudinally compressible portion may also be compressible in other directions as well. In some cases, the compressible portion is resiliently compressible such that the compressible portion returns to substantially its original shape and dimensions when the compressive force is released.

The compressible portion may be designed in various ways to be provided with longitudinal compressibility. In some cases, the compressible portion may have a structure that is longitudinally compressibility (e.g., coil springs or accordion-type pleating). In some cases, the compressible portion may be made of a material that is compressible (e.g., an elastomeric material). The compressible portion may be located anywhere on the proboscis shaft, including the distal end. In some cases, the proboscis shaft may comprise a distal hood, with the compressible portion located on the distal hood.

By having a compressible portion on the proboscis shaft, an operator using the catheter device may rely on visual cues (e.g., by fluoroscopy) instead of tactile sensation to determine the amount of pressure that is being applied against the tissue. As such, in some cases, the proboscis shaft may include one or more radiopaque markers. For example, radiopaque markers may be positioned both proximal and distal to the compressible portion so that the amount of compression can be viewed under x-ray fluoroscopy.

The following non-limiting examples further illustrate various embodiments of the present invention. Referring to FIGS. 8A and 8B, a catheter device 90 comprise a proboscis shaft having a proximal portion 93 and a distal portion 94, a coil spring 96 disposed between proximal portion 93 and distal portion 94 of the proboscis shaft, and an injection needle 20 disposed in the proboscis shaft. FIG. 8A shows coil spring 96 in an uncompressed state, and FIG. 8B shows coil spring 96 in a compressed state.

The proboscis shaft has two radiopaque markers that can be visualized under x-ray fluoroscopy. A proximal radiopaque marker 23 is positioned on proximal portion 93 of the proboscis shaft, and a distal radiopaque marker 24 is positioned on distal portion 94 of the proboscis shaft. When compressive force is applied to the proboscis shaft, the gap between the two radiopaque markers will decrease, which indicated the amount of compression the proboscis shaft is experiencing and/or the amount of force being applied to the tissue surface.

In alternate embodiments, the coil springs does not separate the proboscis shaft into distal and proximal portions, but rather is integrated into the proboscis shaft. For example, the coil spring may be located inside the proboscis shaft, outside the proboscis shaft, or within the thickness of the proboscis shaft wall. In another alternate embodiment, the wall of the proboscis shaft itself may be formed into a coil spring (e.g., by making spiral cuts through the proboscis shaft).

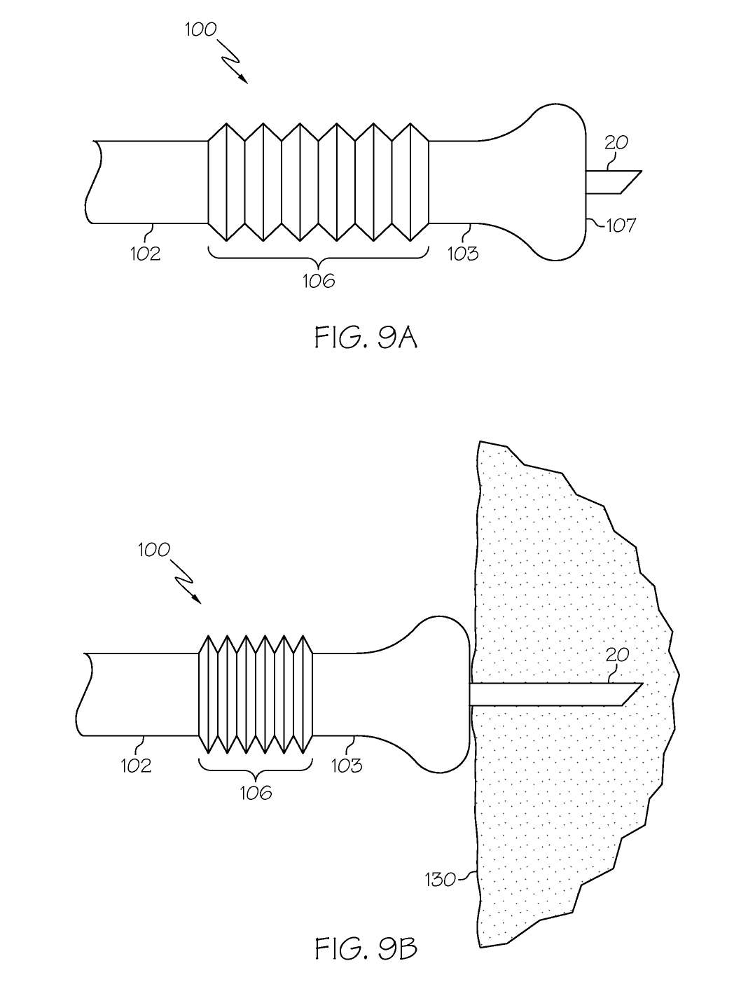

Referring to FIGS. 9A and 9B, a catheter device 100 comprises a proboscis shaft having a proximal portion 102 and a distal portion 103, an accordion-type pleated section 106 between proximal portion 102 and distal portion 103 of the proboscis shaft, and an injection needle 20 disposed within the proboscis shaft. Pleated section 106 may form a single unitary structure with proximal portion 102 and distal portion 103 of the proboscis shaft, or alternatively, pleated section 106 may be a separate unit joining the two portions of the proboscis shaft. FIG. 9A shows pleated section 106 in an uncompressed state, and FIG. 9B shows pleated section 106 in a compressed state.

FIG. 9B also shows the operation of catheter device 100 when used for myocardial injections. The distal end of catheter device 100 is positioned in an internal chamber of the heart and is guided to approach the myocardial wall 130. The distal face 107 of the proboscis shaft is pressed against the myocardial wall 130, causing pleated section 106 to become compressed. Injection needle 20 is made to penetrate the myocardial wall 130. Penetration of the myocardial wall 130 by injection needle 20 may occur before, after, or simultaneous with the proboscis shaft engaging the myocardial wall 130. For example, injection needle 20 may be fully retracted inside the proboscis shaft, and then exposed when pleated section 106 becomes compressed. A therapeutic or diagnostic agent is then delivered to the myocardium through injection needle 20.

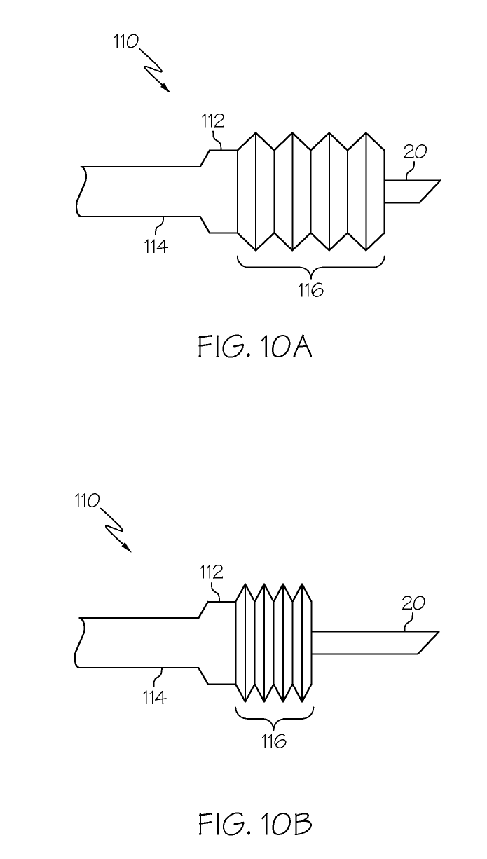

Referring to FIGS. 10A and 10B, a catheter device 110 comprises a proboscis shaft 114 having a hood portion 112. The distal end of hood portion 112 has an accordion-type pleated section 116. FIG. 10A shows pleated section 116 in an uncompressed state, and FIG. 10B shows pleated section 116 in a compressed state. In some cases, pleated sections 106 or 116 resiliently return to their uncompressed condition when the compressive force is released.

The catheter devices of the present invention may have any of various applications in catheter-guided interventions. For example, in addition to myocardial injections, the catheter devices of the present invention may be used for delivering electrical stimulation to the myocardium via electrodes. Also, the catheter devices of the present invention may be used for other target sites in the body, such as the blood vessels, gastrointestinal tract (e.g., stomach, esophagus, small intestine, large intestine), or the genitourinary tract (e.g., bladder, ureters).

The foregoing description and examples have been set forth merely to illustrate the invention and are not intended to be limiting. Each of the disclosed aspects and embodiments of the present invention may be considered individually or in combination with other aspects, embodiments, and variation of the invention. Modifications of the disclosed embodiments incorporating the spirit and substance of the invention may occur to person skilled in the art and such modification are within the scope of the present invention.

What is claimed is:

1. A catheter device comprising:
a proboscis shaft having a lumen and an exit opening at the distal end of the proboscis shaft;
a tissue surface engagement structure positioned at the distal end of the proboscis shaft, wherein the tissue surface engagement structure has a first configuration and a second configuration, and wherein the transverse profile of the tissue surface engagement structure is larger in the second configuration than in the first configuration; and
a proboscis disposed within the lumen of the proboscis shaft.

2. The catheter device of claim 1, wherein the tissue surface engagement structure switches from the first configuration to the second configuration by self-actuation.

3. The catheter device of claim 2, wherein the tissue surface engagement structure is biased towards the second configuration.

4. The catheter device of claim 1, further comprising an elongate tubular member having a lumen and an exit opening at the distal end of the elongate tubular member, and wherein the proboscis shaft is slidably disposed within the lumen of the elongate tubular member.

5. The catheter device of claim 4, wherein the tissue surface engagement structure is maintained in the first configuration when the proboscis shaft is retracted inside the elongate tubular member, and wherein the tissue surface engagement structure switches to the second configuration when the proboscis shaft is advanced out of the elongate tubular member.

6. The catheter device of claim 1, wherein the size of the transverse profile of the tissue surface engagement structure in the second configuration is at least 1.5 times the size of the transverse profile in the first configuration.

7. The catheter device of claim 1, wherein the size of the transverse profile of the tissue surface engagement structure in the second configuration is 1.5 to 10 times the size of the transverse profile in the first configuration.

8. The catheter device of claim 1, wherein the size of the transverse profile of the tissue surface engagement structure in the second configuration is 1.5 to 5 times the size of the transverse profile in the first configuration.

9. The catheter device of claim 1, wherein the size of the transverse profile of the tissue surface engagement structure in the second configuration is 1.5 to 3 times the size of the transverse profile in the first configuration.

10. The catheter device of claim 1, wherein the proboscis is slidable in relation to the proboscis shaft.

11. The catheter device of claim 1, wherein the tissue surface engagement structure is an expandable assembly, and wherein the first configuration is a collapsed configuration and the second configuration is an expanded configuration.

12. The catheter device of claim 11, wherein the expandable assembly is self-expandable.

13. The catheter device of claim 11, wherein the expandable assembly comprises a structure selected from the group consisting of: an umbrella structure, a canopy structure, a basket structure, a wire mesh structure, a tab, a petal, a strip, a sleeve, and a wire loop.

14. The catheter device of claim 1, wherein the tissue surface engagement structure is a hinged assembly comprising one or more hinged members that are hingedly joined to the proboscis shaft at one or more hinge portions, and wherein the first configuration is a closed configuration and the second configuration is an open configuration.

15. The catheter device of claim 14, wherein the hinged assembly comprises a single hinged member, and wherein the single hinged member is a partially transected distal end of the proboscis shaft.

16. The catheter device of claim 14, wherein the hinged assembly comprises a plurality of hinged members.

17. The catheter device of claim 14, wherein the hinged assembly is self-opening.

18. The catheter device of claim 14, wherein the hinged assembly is actuated by compressing the one or more hinged members against the target tissue surface.

19. A method for delivering a therapeutic or diagnostic agent into myocardium, comprising:
positioning the distal end of a catheter device within an internal chamber of the heart, wherein the catheter device comprises:
(a) a proboscis shaft having a lumen and an exit opening at the distal end of the proboscis shaft;
(b) a tissue surface engagement structure positioned at the distal end of the proboscis shaft, wherein the tissue surface engagement structure has a first configuration and a second configuration, and wherein the transverse profile of the tissue surface engagement structure is larger in the second configuration than in the first configuration; and
(c) a proboscis disposed within the lumen of the proboscis shaft; switching the tissue surface engagement structure from the first configuration to the second configuration;
penetrating the myocardium with the proboscis; and
injecting a therapeutic or diagnostic agent into the myocardium through the proboscis.

20. The method of claim 19, wherein the proboscis is slidable in relation to the proboscis shaft, and wherein penetrating the myocardium comprises advancing the proboscis.

21. The method of claim 19, wherein switching the tissue surface engagement structure is performed by self-actuation.

22. The method of claim 19, wherein the catheter device further comprising an elongate tubular member having a lumen and an exit opening at the distal end of the elongate tubular member, and wherein the proboscis shaft is slidably disposed within the lumen of the elongate tubular member.

23. The method of claim 22, wherein switching the tissue surface engagement structure comprises advancing the proboscis shaft such that the tissue surface engagement structure exits the exit opening on the elongate tubular member.

24. The method of claim 19, wherein the tissue surface engagement structure is an expandable assembly.

25. The method of claim 19, wherein the tissue surface engagement structure is a hinged assembly.

26. A catheter device comprising:
a proboscis shaft having a lumen and an exit opening at the distal end of the proboscis shaft;
a deformable cushion positioned at the distal end of the proboscis shaft, wherein the cushion has a pocket filled with reshapeable material, and wherein the cushion has a contact surface for engaging a target site; and
a proboscis disposed within the lumen of the proboscis shaft.

27. The catheter device of claim 26, wherein the reshapeable material is sufficiently viscous that the cushion does not substantially deform under impulse pressures.

28. The catheter device of claim 26, wherein the area of the contact surface when the cushion is deformed under compressive forces encountered during myocardial injection procedures is at least 1.5 times the area of the contact surface when the cushion is undeformed.

29. The catheter device of claim 26, wherein the area of the contact surface when the cushion is deformed under compressive forces encountered during myocardial injection procedures is at least 1.5 to 10 times the area of the contact surface when the cushion is undeformed.

30. The catheter device of claim 26, wherein the area of the contact surface when the cushion is deformed under compressive forces encountered during myocardial injection procedures is at least 1.5 to 3 times the area of the contact surface when the cushion is undeformed.

31. The catheter device of claim 26, wherein the cushion substantially returns to its original shape and dimensions when a compressive force is released.

32. The catheter device of claim 26, further comprising an elongate tubular member having a lumen and an exit opening at the distal end of the elongate tubular member, and wherein the proboscis shaft is slidably disposed within the lumen of the elongate tubular member.

33. The catheter device of claim 26, wherein the reshapeable material is selected from the group consisting of: a fluid, a liquid, a gas, a gel, and a foam material.

34. A method of delivering a therapeutic or diagnostic agent into myocardium, comprising:
positioning the distal end of a catheter device within an internal chamber of the heart, wherein the catheter device comprises:
(a) a proboscis shaft having a lumen and an exit opening at the distal end of the proboscis shaft;
(b) a deformable cushion positioned at the distal end of the proboscis shaft, wherein the cushion has a pocket filled with a reshapeable material; and
(c) a proboscis disposed within the lumen of the proboscis shaft
engaged the cushion with the myocardial wall to create a contact surface between the cushion and the myocardial wall;
penetrating the myocardium with the proboscis;
compressing the cushion such that the area of the contact surface increases; and
injecting a therapeutic or diagnostic agent into the myocardium through the proboscis.

35. The method of claim 34, wherein the contact surface has an area of at least 0.9 mm².

36. The method of claim 34, wherein the area of the contact surface when the cushion is deformed under compression is at least 1.5 times the area of the contact surface when the cushion is undeformed.

37. The method of claim 34, wherein the area of the contact surface when the cushion is deformed under compression is from 1.5 to 10 times the area of the contact surface when the cushion is undeformed.

38. The method of claim 34, wherein the area of the contact surface when the cushion is deformed under compression is from 1.5 to 3 times the area of the contact surface when the cushion is undeformed.

39. The method of claim 34, wherein the reshapeable material is sufficiently viscous that the cushion does not substantially deform under impulse pressures.

40. The method of claim 34, wherein the proboscis is fully retracted inside the proboscis shaft, and wherein compressing the cushion exposes the proboscis.

41. A catheter device comprising:
a proboscis shaft having a longitudinally compressible portion, a lumen, and an exit opening at the distal end of the proboscis shaft; and
a proboscis disposed within the lumen of the proboscis shaft.

42. The catheter device of claim 41, wherein the compressible portion comprises a coil spring.

43. The catheter device of claim 41, wherein the compressible portion comprises accordion-type pleats.

44. The catheter device of claim 41, wherein the proboscis shaft further comprises a distal hood, and wherein the compressible portion is located on the distal hood.

45. The catheter device of claim 41, wherein the proboscis shaft includes at least one radiopaque marker.

46. The catheter device of claim 45, wherein the radiopaque marker is positioned proximal to the compressible portion and another radiopaque marker is positioned distal to the compressible portion.

47. The catheter device of claim 41, wherein the compressible portion is resiliently compressible.

48. The catheter device of claim 41, further comprising an elongate tubular member having a lumen and an exit opening at the distal end of the elongate tubular member, and wherein the proboscis shaft is slidably disposed within the lumen of the elongate tubular member.

49. A method for delivering a therapeutic or diagnostic agent into myocardium, comprising:
positioning the distal end of a catheter device within an internal chamber of the heart, wherein the catheter device comprises:
(a) a proboscis shaft having a longitudinally compressible portion, a lumen, and an exit opening at the distal end of the proboscis shaft; and
(b) a proboscis disposed within the lumen of the proboscis shaft.
engaging the proboscis shaft with the myocardial wall;
penetrating the myocardium with the proboscis; and
injecting a therapeutic or diagnostic agent into the myocardium through the proboscis.

50. The method of claim 49, wherein engaging the proboscis shaft with the myocardial wall causes compression of the compressible portion.

51. The method of claim 49, wherein the compressible portion comprises a coil spring.

52. The method of claim 49, wherein the compressible portion comprises accordion-type pleats.

53. The method of claim 49, wherein the proboscis shaft includes at least one radiopaque marker.

54. The method of claim 53, wherein the radiopaque marker is positioned proximal to the compressible portion and another radiopaque marker is positioned distal to the compressible portion.

55. The method of claim 50, wherein the proboscis is fully retracted inside the proboscis shaft, and wherein compression of the cushion exposes the proboscis.
